(19) United States
(12) Patent Application Publication
(10) Pub. No.: US 2012/0209214 A1
(43) Pub. Date: Aug. 16, 2012

(54) Pocket douche upright position

(76) Inventors: Russell Uy, Daly City, CA (US); Liberty Bernardino Uy, Daly City, CA (US)

(21) Appl. No.: 12/931,933

(22) Filed: Feb. 15, 2011

(51) Int. Cl. A61M 3/02

(52) U.S. Cl. 604/212

(57) ABSTRACT

The Upright Pocket douche Syringe will hermetically attach to standard bottled water. The nozzle's rear pipe has an opening with a slightly similar dimension to the bottle neck of standard bottled water that is available in the market. Attached to the cap chamber is the rear pipe with an axial passage in fluid communication to the nozzle. The rear pipe has thin rings/threads around the pipe passage with diameters slightly larger than the neck opening of the water bottle it will be attached to, so that when the rings are pushed into the water bottle, it hermetically joints the two. It then provides a smooth flow of water spraying out from the nozzle holes use for douche or enema.

### BACKGROUND OF THE PRESENT INVENTION

Vaginal irrigation devices have gained wide use in cleansing and treating the female organs. One such system previously known reusable douche have been particularly inconvenient for the user when away from home due to the bulkiness of the equipment and the maintenance of cleaning the device due to the frequent lack of facilities necessary to perform these operations, especially when traveling.

Disposable douches are also inconvenient to use due to its bulkiness and the need to assemble many parts during use.

In addition to the disadvantages of previous inventions, most disposable pre-mix douches are not allowed on airplanes because of the evolving security threats to aviation. It is also too bulky to put inside the purse.

### SUMMARY

This invention may be summarized at least in part with reference to its objective. It is the primary objective of the present invention to provide the most convenient, effective, sanitary, safe to carry, and handy way to douche conveniently without the bulkiness of the kit. All you need to carry is the present invention. When you are ready to use it, just connect it to the any standard bottled water.

The present invention can be manufactured as a soft flexible material to allow the user to adjust the syringe to the most comfortable position to avoid discomfort or injury. Therefore, it doesn't limit the manufacturing material to plastic.

Also, this invention can simply be produced, providing inexpensive syringes for healthy vaginal hygiene.

### DRAWING

### Figures

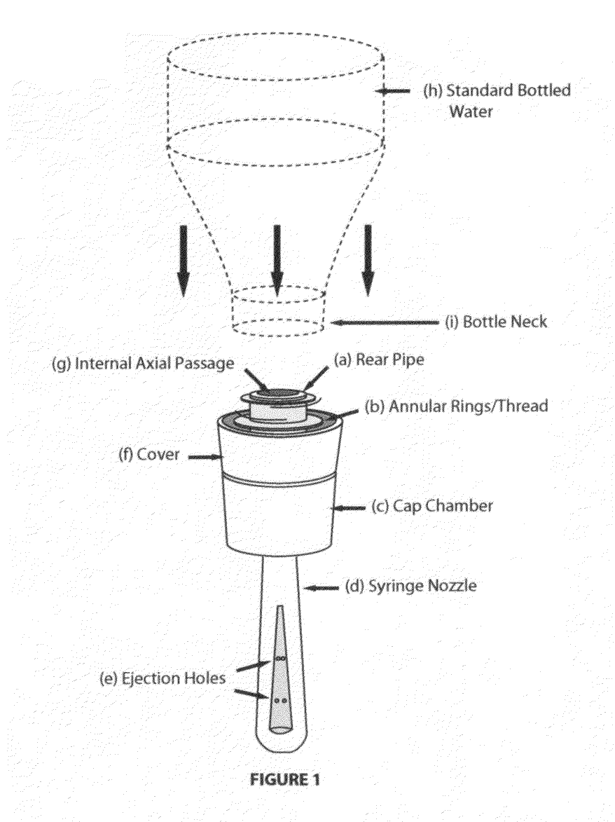

FIG. 1 is the Perspective View of a syringe for vaginal irrigation in accordance with the preferred embodiment of the present invention. It shows combined syringe, for fitting on bottled water, showing my new design. (The bottled water illustrated as broken lines is not part of the claimed design)

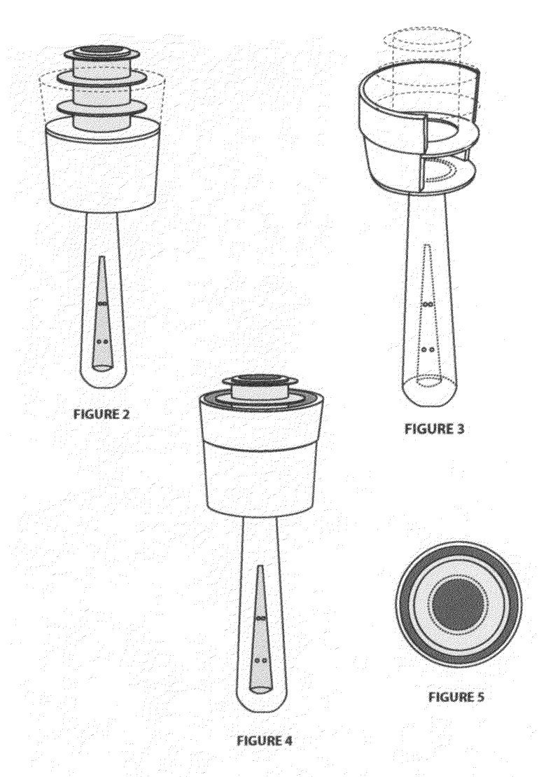

FIG. 2 The Perspective View of the syringe without the cover of FIG. 1

FIG. 3 The Exploded View of the syringe of FIG. 1

FIG. 4 The Side View of the syringe of FIG. 1

FIG. 5 The Top View of the Syringe of FIG. 1

### DRAWINGS

### Reference

a. Rear Pipe
b. Annular Rings/Thread
c. Cap Chamber
d. Syringe/Nozzle
e. Ejection Holes
f. Cover
g. Internal Axial Passage
h. Standard Bottled Water
i. Bottle Neck

### DETAILED DESCRIPTION OF THE INVENTION

Referring to the drawings and particularly FIG. 1, the preferred embodiment of the invention, generally designated FIG. 1—A & B is illustrated that the Rear Pipe and Annular Rings/Thread is provided for sealing the syringe to any bottled water. The cap chamber (c) has a rear pipe (a). The rear pipe (a) has plurality of thin, resilient annular rings (b) affixed around it that seals the neck (i) of any bottled water (h). The rear pipe (a) has internal axial passage (g) through which the fluid will pass from the bottled water (h) to the ejection holes (e). When the rear pipe (a) of the cap chamber (c) is forced into the neck (i) of bottled water (h) the annular rings/thread (b) are compressed against the inside surface of the neck (i) of bottled water (h) and sealing the internal axial passage (g). The syringe/nozzle (d) comprises a hollow vaginal insert. When the present invention is completely connected to the neck (i) of the standard bottled water (h), the syringe (d) is now ready to be inserted into the vagina. When bottled water (h) is compressed by hand, the water due to pressure flows from standard bottled water (h) to the internal axial passage (g), to the cap chamber (c) to the syringe/nozzle (d), then flows through the ejection holes (e). Suitable pressure may then be applied to provide the desired flow rate of water until the standard bottled water (h) is empty. The syringe (d) may be then be removed and disposed.

As described above, the present invention is only one piece, which has a pipe that is inserted inside the neck of bottled water to seal it. The rear pipe has a plurality of thin, resilient annular rings affixed around it. When the rear pipe of the cap chamber is forced into the neck of bottled water the annular rings/threads are compressed against the inside surface of the neck of bottled water and sealing the internal axial passage. When the present invention is completely assembled to the neck of the standard bottled water, the syringe is now ready to be inserted into the vagina. When bottled water is compressed by hand, the water due to pressure flows from standard bottled water to the internal axial passage, to the cap chamber to the syringe/nozzle, then flows through the ejection holes. Suitable pressure may then be applied to provide the desired flow rate of water until the standard bottled water is empty. The syringe may be then be removed and disposed.

While a full and complete description of the invention has been set forth in accordance with the dictates of the Patent Statues, it is to be understood that the invention is not intended to be limited to be specific from herein shown.

Accordingly, modification of the invention may be shown without departing from the spirit hereof or the scope of the accompanying claims.

1. A Upright Syringe for vaginal irrigation, compromising:
a. The upright vaginal syringe is one piece which has a cap, pipe and syringe
b. The cap has a pipe and it has plurality of thin, resilient annular rings affixed around the tube
c. The pipe has internal axial passage through which the fluid will pass from the bottled water to the syringe
d. The resilient annular rings/threads has a greater dimension than the inside diameter of the neck container
e. When the pipe is forced into the neck of the bottled water the rings/threads are compressed against the inside surface of the neck container, and sealing the junction between the pipe cap syringe and the bottled water
f. The Claim 1 can be manufactured in Plastic (polyethylene); however, it is not limited to plastic and can be manufactured in rubber or in any other materials.
g. The irrigation nozzle/syringe is provided with two ejection holes on the side however, it can also be manufactured with plurality of apertures.

2. The FIG. 8 shows that claim 1 can be manufactured solely as a connecting pipe without the outer cover (FIG. 1-f)

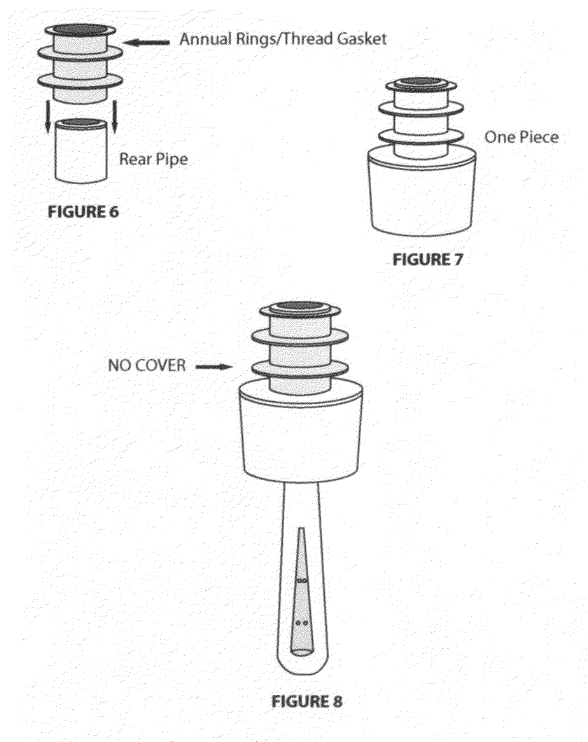

3. FIG. 6 shows that the claim 1 Annular Rings/thread Gasket and Rear Pipe can be manufactured as separate parts or FIG. 7 shows that the claim 1, Annular Rings/Thread and the Rear Pipe can be molded as one piece.
